(19) United States
(12) Patent Application Publication
(10) Pub. No.: US 2009/0030266 A1
(43) Pub. Date: Jan. 29, 2009

(54) Medical Device to Exercise Urogenital Muscles

(76) Inventor: Margaret Phillipa Treanor, New South Wales (AU)

Correspondence Address: Margaret Phillipa Treanor, 1 Olympic Court, Carlingford, 2118, AU

(21) Appl. No.: 12/224,742

(22) PCT Filed: Jan. 11, 2007
(86) PCT No.: PCT/AU2007/000011

§ 371 Date: Sep. 5, 2008

(30) Foreign Application Priority Data
Mar. 10, 2006 (NZ) 535186

(51) Int. Cl. A61F 2/02; A61N 1/00

(52) U.S. Cl. 600/30; 607/138

(57) ABSTRACT

A medical device being an insert comprising a shaft and a head and a plurality of spring loaded domes which in use act as electrodes to convey an electrical current via a manual dome retractor to a female subject; uses electronic stimulation to exercise and strengthen to tone the muscles of the pelvic floor, urogenital triangle and tighten the intravaginal cavity. The device also has the capability to treat medical and sexual problems related to these areas and supersedes the manual pelvic floor exercises and need for the husband stitch.

### THE FIELD OF THE INVENTION

(c) This invention is a medical device to electronically force the muscles of the pelvic floor, vagina and muscles of the urogenital triangle to contract and release, causing the muscles to exercise.

This device would replace the need for pelvic floor exercises and the husband stitch. The term husband stitch is used to describe an additional suture to tighten the vagina post childbirth and husbands usually directed the request to the administering doctor at the time of labour. This device would tone and tighten the vaginal muscles of the intravaginal cavity, strengthen the pelvic floor and urogenital triangle muscles by electronically exercising the area through electronic stimulation. Electronic stimulation has been readily available for many purposes and has proven to be a safe and effective method of treatment for many medical problems. This medical device electronically forces the contracting and releasing of the muscles which allows the user to relax, using no conscious effort to hold a contraction whilst exercising this area. It is small and compact making it portable and discreet allowing the user to have more dignity, privacy and control over these areas. The device has simplistic instructions, which allows the user to control the usage and dosage, making it user friendly and needing no previous medical background or professional help. Also has capability to cure and prevent medical problems associated with weak pelvic floor muscles like incontinence.

### PRIOR ART

A previous attempt U.S. Pat. No. 6,086,549 to overcome some of the medical aspects was to provide a probe with ×2 bands surrounding an electrode shell of an electrode carrier being part of the probe. These were used to electronically stimulate the pudendal nerve via the intravaginal cavity to treat incontinence. This solution has had some disadvantages. A handle was needed to place the probe within the intravaginal cavity. The probe had ×3 alternative electrode carriers, which catered for the varying depths and size of the intravaginal cavity. The bands also relied on the width of the electrode carrier to retain the probe within the intravaginal cavity and converge with the walls of the intravaginal cavity for sufficient electrical stimulation. The tip of the head of the probe had no electrodes to convey an electrical charge to electronically stimulate the pelvic floor directly, but relied on the stimulation of the pudendal nerve to treat the incontinence.

U.S. Pat. No. 5,662,699 relied on a collapsible airtight sheath with a resilient skeleton which relied on air pumped into the sheath to expand enough for the outer conductive bands to relay the electrical charge to the intravaginal walls. U.S. Pat. No. 4,296,760 also has an inflatable elongated element which comprises of a bladder prepared from an expandable material such as rubber to retain the device within the intravaginal cavity. Both of these patents rely on air to expand the carrier for the electrodes to make contact with the vaginal wall. Patent number FR2754717A1 has an elongated carrier with a disposable sheath with two electrodes as part of the sheath that also aligns with rods that are connected to a band and used as electrodes of the carrier that extend to relay the electrical charge to the vaginal walls. The endings of the rod are detachable and make the device questionable as to whether these endings would become detached while in use. U.S. Pat. No. 5,046,511 relies on a removable pad fastened by conductive snap fasteners and wrapped around and secured to a carrier before insertion into the vagina. The pad could easily become undone during insertion. European patent number 0411632A1 relies on a nonconductive sheet of material with conductive electrode elements on the exterior surface of the sheet formed into a diametrically compressible spiral tending to unwind within the vagina, retaining the device. This device relies on the user to hold the vaginal electrode by hand to maintain the compressed spiral state for insertion into the vagina.

### OBJECTS OF THE INVENTION

The object of the present invention is to provide an insert that will in some ways prevent the above disadvantages, and utilise existing technology to replace pelvic floor exercises and the need for the husband stitch. The device uses spring-loaded domes in an active state to retain the device whilst making contact with the intravaginal cavity and pelvic floor. This enables the device to relay an electrical current to these areas to electronically exercise the muscles. Because of this there is no need to change the electrode carriers to fit the contours of the intravaginal cavity. The device is long enough to insert into the vagina without any other assistance like a handle. The domes are strategically placed to allow stimulation directly to the vagina, pelvic floor and urogenital muscles. Electronically forcing the muscles of the vagina, pelvic floor and urogenital triangle to contract and release causes the muscle to exercise without any conscious effort by the user to hold the contraction. This would forcefully strengthen tone and tighten these areas for prevention of any future medical and sexual problems associated with these areas. The basic design and technology of the device makes it simplistic and user friendly and the compact nature of the device enables it to be used discreetly and privately needing no professional help or instruction. This would allow the user to have more dignity and control over these areas as they age.

### BRIEF DESCRIPTION OF THE DRAWINGS

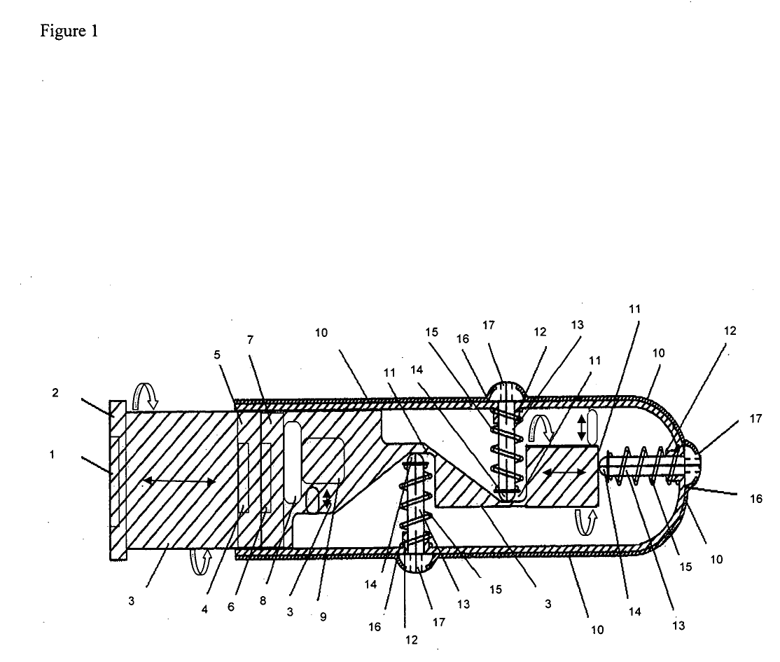

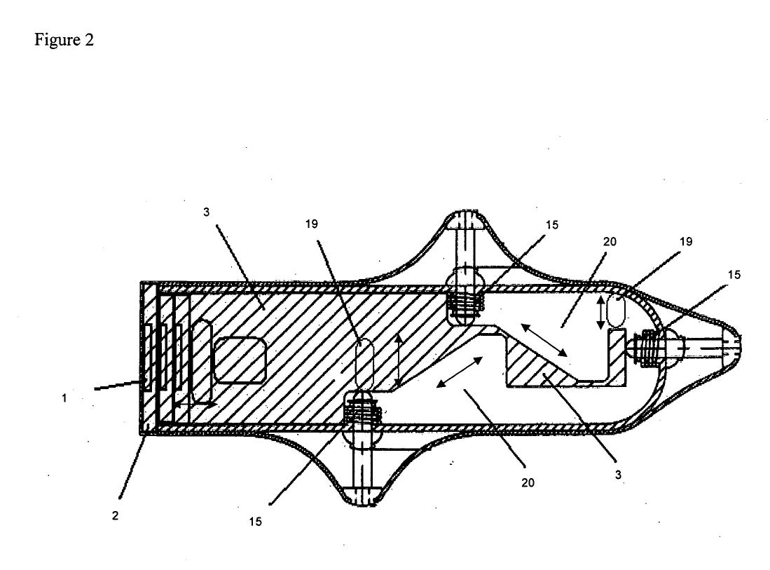

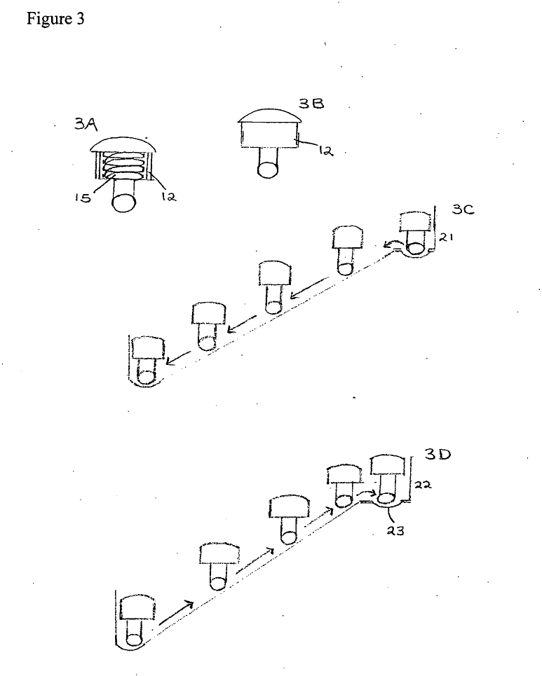

One preferred form of the invention will now be described with reference to the accompanying drawings, of which; FIG. 1 shows the medical device assembled as one unit in an inactive form according to the invention, shown as separate items; FIG. 2 shows the same device in an active form. FIGS. 3,4,5 and 6 show the components and processes of the device whilst active and inactive.

### DETAILED DESCRIPTION OF THE INVENTION

In the form shown, it is an insert, cylindrically shaped, which fits comfortably within the intravaginal cavity as shown in FIG. 1. The device as shown split in half in FIG. 1 also consists of a head, shaft and base. The outer layer of the device consists of non-conductive flexible material 10. The head and shaft of the insert has a number of spring-loaded domes each made of conductive material 17, the dome springs 15 are housed within the dome shell 12 and connected at the base of each dome spring is a rigid connector consisting of conductive material 13 with a rotating ball at the opposite end 14 also consisting of a conductive material. This is to help with the expansion, retraction, mobility and electrical conductivity to and of the domes; each dome head is surrounded by non-conductive flexible material. The domes are the electrodes of the device and are controlled by the manual dome retractor 3 to expand to touch the walls of the intravaginal cavity.

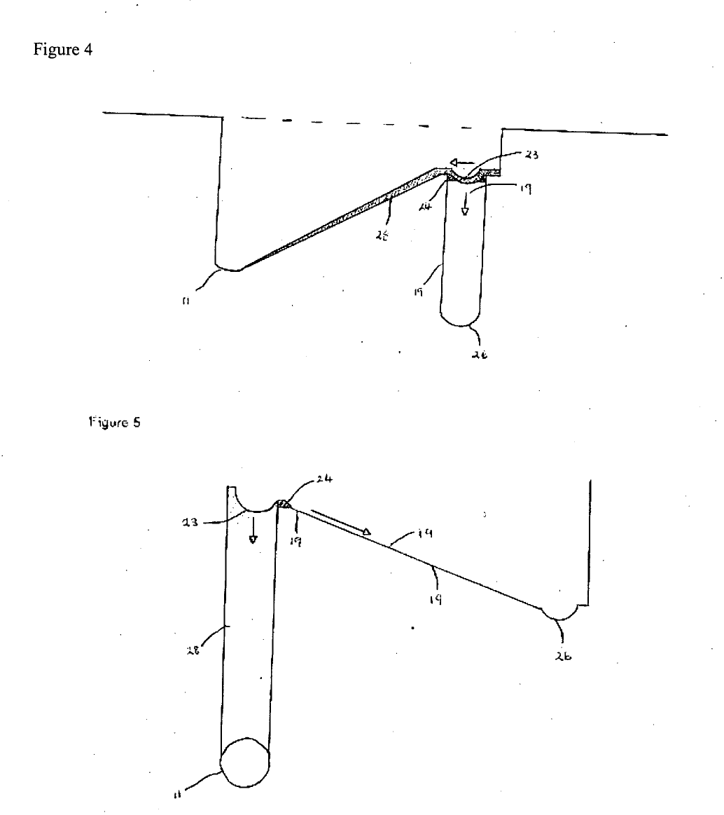

The manual dome retractor also shown in FIG. 2, number 3 consists of non conductive rigid material that is a movable sleeve within the device, controlled by a control device to manually turn the medical device on and off 2. The control device to turn the medical device on and off can move up and down and side to side guided by the grooves of the ramps 19 and 20. This process is also automated by a second control device 1 that works in conjunction with the control device to manually turn the device on and off. Active ramps as shown in FIGS. 4 and 5 are lined with conductive material 28; when the balls of the connectors align with the grooves of the active ramp they not only control the movement of the domes but also convey the electrical charge to the domes, bringing them from an inactive to an active state and vice versa.

In an inactive state as shown in FIG. 1 all the connectors sit within the seat of the home base 11, the springs are in a relaxed state 15 and the dome heads sit within the external shell of the device; the dome seat 16.

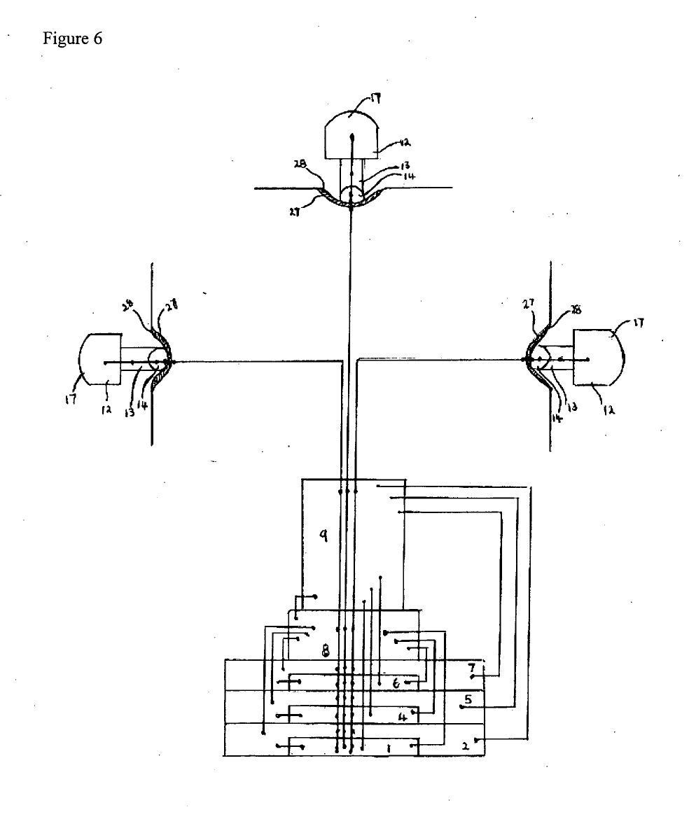

When activated the manual dome retractor moves in an upward motion as shown in FIG. 3D, number 22, forcing the connectors to travel up the incline of the active ramp until seated within the active base 23. Pressured by the manual dome retractor the connectors force the springs and then the domes to expand as shown in FIG. 2, number 15 to touch the walls of the intravaginal cavity and pelvic floor. The electrical current is passed from the power source as shown in FIG. 6, number 9 and conveyed by the wiring to the internal side of the manual dome retractor number 27, to the conductive lined grooves 28 on the external side of the manual dome retractor. Electricity is then passed through the dome balls 14, then dome connectors 13 and dome springs encased by the dome shell 12, to the head of the domes 17; the electrodes and then to the walls of the intravaginal cavity.

The retraction ramps as shown in FIGS. 4 and 5 number 19 form part of the manual dome retractor and are activated by the control device that turns the medical device on and off. The retraction ramps are non conductive grooves within the manual dome retractor to manually retract the domes, if the unit malfunctions during use. The connectors and domes are first aligned to the retraction ramp through the active base 23 which is the point of entry to both ramps. This is activated by turning the manual dome retractor anticlockwise past the off stage of the control device to turn the device on and off. The connector is then forcefully pressured to jump the safety lip 24 and to follow down the ramp 19, into the seat of the retraction ramp 26 also shown in FIG. 1 number 26 and FIG. 3C number 21. The domes are inactive and are manually withdrawn into the medical device itself. The device is then withdrawn from the intravaginal cavity.

Once the device is withdrawn from the intravaginal cavity, the domes and connectors need to be returned to the home base FIGS. 4 and 5, number 11. The manual dome retractor is turned clockwise before the on stage of the device. This will reverse the process returning the domes and connectors to the active base stage as shown in FIGS. 4 and 5 number 23 causing the inactive domes to extend manually (can be cleaned by this method too) as they go up the retraction ramp as shown in FIG. 3D, number 22. Then pushing the base of the manual dome retractor upwards as shown in FIG. 1 number 3 (which is also the control device to turn the device on and off) into the device will enable the domes and connectors to travel up the ramp as shown in FIG. 3D, number 22 to the active base 23, then aligning the connector to the active ramp as shown in FIG. 3C, number 21 and pulling the manual dome retractor base out as shown in FIG. 1, number 3 will encourage the connector to follow the decline of the ramp, as shown in FIG. 3C number 21 returning it to the home base which is non conductive, as shown in FIGS. 4 and 5 number 11 to its inactive state, ready for use again.

The electrodes: the domes are oval in shape made of conductive material and house the dome springs as shown in FIG. 3A numbers 15 and encased by the dome shell 12, also shown in FIG. 3B number 12. These are strategically placed around the head and length of the shaft and surrounded by non-conductive rubber.

The base of the insert protrudes from the vulva. The base consists of a control device to turn the power of the device on and off and to control the manual dome retractor which moves up and down and side to side. This movement controls the expansion and retraction of the domes as shown in FIG. 1 number 3. A second control device that automates this process and works in conjunction with the control device to turn the device on and off is shown in FIG. 1 number 1.

A third control device as shown in FIG. 1 number 5 at the base of the medical device is controlled by the user to regulate the intensity of the electrical charge, 0=OFF, 1=lowest intensity of electrical charge to 5=highest intensity (strongest contraction) and works in conjunction with a fourth control device as shown in FIG. 1 number 4 to regulate and direct the electrical charge. A fifth control device 7 controls the time the contraction lasts 0=OFF, 1=shortest time contraction lasts, 5=longest time contraction held and can include and works in conjunction with sixth control device 6 to automatically break the electrical current, which then causes the muscles to relax, however this can also be controlled manually. The device also includes a seventh control device 8 that automatically switches off the unit if it detects any abnormal function like heat, or sparking in the unit or the components of the unit.

The base and manual dome retractor also houses the power source, a plurality of cells 9, which supplies the electrical charge for the device.

FIG. 6 shows the relativity of the interrelationships between the components of the device. The lines show the path of the insulated wiring which is the conveyor of the electrical charge from the power source to the domes via all the components, this will also show how each component affects each other. The power source 9 supplies the power for the whole unit and works in conjunction with every single component while the device is active. A control device to turn the device on and off 2 and also manually retracts and extends the spring loaded domes also works in conjunction with a second control device 1 that automates the process of turning the device on and off and automatically extends and retracts the spring loaded domes. This also affects the manual dome retractor itself and also every component of the device by activating the power supply. A third control device controls the intensity of the electrical charge 5 and a fourth control device regulates and directs the electrical charge 4; they work in conjunction with each other. A fifth control device controls the contraction length 7 and a sixth control device automatically breaks the electrical current 6; they work in conjunction with each other. A seventh control device 8 is electronically linked to each mechanical and electronic components of the device to monitor the device for any abnormal activity such as over heating or sparking of components within the device.

The control devices 2, 5, and 7 work in conjunction with the control device 8 and the internal base of the grooves 27 to convey the electrical charge to the external base of the manual dome retractor 28. This enables the seventh control device 8 to monitor the safe activity of the domes components. The external conductive lined base of the grooves as shown in 28 relay the electrical charge to the dome balls 14, dome connectors 13, and the springs encased by the dome shell 12 to the dome heads themselves 17; all work together to convey the electrical charge to the intravaginal cavity.

After insertion during active use of the device FIG. 2, the spring-loaded domes expand to touch the walls of the intravaginal cavity and pelvic floor which retains the insert while transmitting the electrical charge within the intravaginal cavity. There is no need to adjust the shaft or head to the varying sizes of the intravaginal cavity. The electrical charge, controlled by the dials is relayed to the intravaginal walls through the metals domes forcing them to contract and release, this forces the muscles of the pelvic floor, vagina and urogenital triangle to exercise, to tone and strengthen and tighten these areas.

### Description of the Operation of the Invention

In operation, during active use, the device uses the dials to control the electrical current from the power source, the plurality of cells. The electrical current is relayed via the insulated wiring to the domes of the insert, which are the electrodes. The electrical current is then relayed from the domes, which during active use converges with the contours of the intravaginal cavity walls and pelvic floor. The electrical current causes the muscles of the vagina and pelvic floor to contract. There are several control devices to manage the domes, manual dome retractor and electrical current of the device. A control device to turn the device on and off which is also automated by a second control device. A third control device to control the intensity of the electrical charge and a fourth control device to regulate and direct the charge. A fifth control device is used to control the length of the hold of the contraction and a sixth control device preset to automatically intermittently breaks the electrical current. A seventh control device is used to monitor the safety of the whole device. The electronically controlled contracting and relaxing of the muscles causes them to exercise. A manual dome retractor has also been added to allow the user to manually retract the domes for safety reasons and expand the domes for cleaning purposes.

It will be appreciated that the invention broadly consists in the parts, elements and features described in this specification, and is deemed to include any equivalents known in the art which, if substituted for the described integers, would not materially alter the substance of the invention

What I claim is:

1. A medical device being an insert comprising a shaft and a head and a plurality of spring loaded domes which in use act as electrodes to convey current to a female subject using electronic stimulation; an electrical charge is relayed through the device and is controlled by a control device to manually turn the device on and off and manually extends and retracts spring loaded domes; a second control device automates the process of turning the device on and off, and also automates the extension and retraction of the spring loaded domes; a third control device that controls the intensity and amount of the electrical charge works in conjunction with a fourth control device that regulates and directs the strength and flow of the electrical charge; a fifth control device that controls the length of time of the contraction, which can also be manually operated to break the electrical current; works in conjunction with a sixth control device that is optional and preset to automatically intermittently break the electrical charge transmitted; a manual dome retractor; a non conductive sleeve within the device has a series of ramps with conductive and non conductive lined grooves used to promote the manual and automatic retraction and expansion of the spring loaded domes; insulated wiring relays electricity to the conductive lined grooves of the ramps to conductive dome balls and connectors to the conductive spring loaded domes which relay the electrical charge to heads of the spring loaded domes; the expansion of the spring loaded domes and the manual dome retractor are controlled by the first and second control devices to turn the device on and off and the grooves within the manual dome retractor; the domes are strategically placed along the shaft and the head of the device which when in use expand to touch the walls of the intravaginal and pelvic floor; the electrical charge is relayed through the head of the domes to the walls of the intravaginal muscles and pelvic floor causing them to repeatedly and automatically contract and relax, exercising the muscles; a seventh control device is used to monitor any abnormal activity of the device such as overheating or sparking within the device and is electronically linked to all mechanical and electronic components of the device, and works in conjunction with all components of the device.

2. The device as claimed in claim 1 has a device that detects abnormal function or activity within the insert and automatically switches off the device.

3. The device as claimed in claim 1 has ramps forming part of the manual dome retractor for manual and automatic retraction and expansion of the domes whilst active and inactive.

4. The device as claimed in claim 1 is cylindrical in shape and has non-conductive rubber surrounding the entirety of the surface of the head, shaft and circumference of the domes, but not the surface of the domes themselves.

5. The device as claimed in claim 1 has the spring loaded domes which in an active state conform to the walls of the vagina, and retains the device within the intravaginal cavity.

6. The device as claimed in claim 1 is powered by plurality of cells and transmits the electrical charge through the device in an active state.

7. The device as claimed in claim 1 has wiring that is insulated with a non conductive material and conveys the electrical charge through the device.

8. The medical device as described and illustrated in the accompanying drawings.
